(19) United States
(12) Patent Application Publication
(10) Pub. No.: US 2013/0309210 A1
(43) Pub. Date: Nov. 21, 2013

(54) ACCELERATION OF HEMATOPOIETIC RECONSTITUTION BY PLACENTAL ENDOTHELIAL AND ENDOTHELIAL PROGENITOR CELLS

(71) Applicant: Regen BioPharma, Inc, La Mesa, CA (US)

(72) Inventor: Thomas Ichim, San Diego, CA (US)

(73) Assignee: Regen BioPharma, Inc, La Mesa, CA (US)

(21) Appl. No.: 13/897,735

(22) Filed: May 20, 2013

Related U.S. Application Data
(60) Provisional application No. 61/648,898, filed on May 18, 2012.

(51) Int. Cl. A61K 35/50

(52) U.S. Cl. CPC A61K 35/50
USPC 424/93.7; 435/325; 435/381

(57) ABSTRACT

Compositions useful for treatment of patients needing hematopoietic stimulation. In one embodiment patients are administered a cellular mixture derived from allogeneic placenta, said cellular mixture comprising substantially of endothelial cells and endothelial progenitor cells.

### CROSS-REFERENCE TO RELATED APPLICATIONS

This application claims priority to U.S. Provisional Application No. 61/648,898, filed May 18, 2012, and entitled “Acceleration of Hematopoietic Reconstitution by Placental Endothelial and Endothelial Progenitor Cells”, which is hereby expressly incorporated by reference in its entirety.

### FIELD OF THE INVENTION

Disclosed are compositions useful for treatment of patients needing hematopoietic stimulation. In one embodiment patients are administered a cellular mixture derived from allogeneic placenta, said cellular mixture comprising substantially of endothelial cells and endothelial progenitor cells.

### BACKGROUND

This application relates to the field of stem cell biology, cell culture, and hematopoietic stimulation. In particular, the invention relates to the area of adjuvant therapies for hematopoietic reconstitution, more specifically, the invention relates to placental cellular populations and products thereof derived from the placenta that are useful for regenerative applications.

Endothelial cells have been previously shown in the art to stimulate hematopoietic reconstitution. Endothelial progenitor cells (EPC) have been shown to possess various regenerative abilities. Despite some data showing autologous/syngeneic endothelial cells having ability to accelerate hematopoietic reconstitution, these cells are not practical for clinical use. Compared to EPC or endothelial cells found in younger tissue such as placenta.

### SUMMARY

Embodiments herein are directed to a composition useful for accelerating reconstitution of the hematopoietic compartment, said composition comprising cells isolated from placenta. Said cells isolated from placenta can be endothelial cells. Said cells isolated from placenta can be isolated from placenta perivascular tissue. Cells isolated form placenta perivascular tissue can expresses a marker selected from a group of markers consisting of: CD144, CD105, and CD31. The placenta perivascular tissue can be isolated from fetal vascular lobules of a hemochorial placenta. Said cells can be prepared by: a) dissociating fetal vascular lobules from a full-term human placenta; b) successively digesting the homogenized lobules of step a) with a preparation of about 2% collagenase, about 0.25% trypsin and about 0.1% DNAse in tissue culture medium; c) filtering the digestion product of step b) to remove particulates; d) obtaining a mononuclear cells from the filtered digestion product of step c) by density gradient centrifugation; e) plating the mononuclear cells on a collagen I-coated tissue culture plate; f) growing the mononuclear cells to confluency; g) detaching the confluent cells from the plate; and h) sorting the detached cells for expression of CD144 and substantially lack of expression of CD45. Said cells isolated by enzymatic digestion of the placenta can be administered into a patient in need of treatment without an expansion step. Said cells isolated by enzymatic digestion of placenta can be grown in a media that allows stem cell proliferation and differentiation activity in vitro. Said cells isolated by enzymatic digestion of placenta substantially lack expression of a marker selected from a group of markers consisting of: CD14, CD34, CD38 and CD45. Said cells can form capillary-like tubules when plated on a Matrigel substrate. Said cells can take up DiI-acetylated-lowdensity-lipoprotein. Said cells can be cultured in a media selected from DMEM, RPMI, EMEM, Iscove's Media, and Ham's F12 media. Said cells can be cultured in a media containing fetal calf serum. Said fetal calf serum can be added to said media at a concentration ranging from approximately 2% to approximately 20%. Said fetal calf serum can be added to said media at a concentration of approximately 10%. Said cells can constitute a population of cells containing endothelial progenitor cells.

Additional embodiments are directed to a composition useful for acceleration of hematopoietic reconstitution, said composition comprising of endothelial cells and endothelial progenitor cells derived from fetal vascular lobules of a hemochorial placenta. Said endothelial progenitor cells can substantially express at least one marker selected from: CD144, CD105, and CD31. Said endothelial progenitor cells can lack substantial expression of a marker selected from a group of markers comprising of: a) CD14; and b) CD45. Said endothelial progenitor cells can form capillary-like tubules when plated on a Matrigel substrate. Said endothelial progenitor cells can be capable of taking up DiI-acetylated-low-density-lipoprotein. Said endothelial precursor cells can be manufactured by: a) dissociating fetal vascular lobules from a full-term human placenta; b) successively digesting the homogenized lobules of step a) with a preparation of about 2% collagenase, about 0.25% trypsin and about 0.1% DNAse in tissue culture medium; c) filtering the digestion product of step b) to remove particulates; d) obtaining mononuclear cells from the filtered digestion product of step c) by density gradient centrifugation; e) plating the mononuclear cells on a collagen I-coated tissue culture plate; f) growing the mononuclear cells to confluency; g) detaching the confluent cells from the plate; and h) sorting the detached cells for expression of CD144 and substantially lack of expression of CD45.

Said compositions herein can be administered intravenously prior to, at the moment of, or subsequent to exposure to an agent or plurality of agents causing destruction of hematopoietic tissue. Said compositions can be administered together with a growth factor capable of stimulating proliferation and/or differentiation of hematopoietic stem cells. Said growth factor can be selected from a group of growth factors selected from the group consisting of: a) G-CSF; b) M-CSF); c) GM-CS; d) stem cell factor; e) IL-1; f) IL-6; g) thrombopoietin; h) IL-7; and i) PDGF.

Additional embodiments include a method of augmenting hematopoiesis in a patient, said method consisting of: a) selecting a patient in need of therapy; b) obtaining a population of cells containing allogeneic placental derived endothelial progenitor cells; and c) infusing into said patient said population of allogeneic placental derived endothelial progenitor cells. According to further embodiments, the patient in need of therapy suffers from a disorder selected from a group of disorders consisting of: a) acute radiation syndrome; b) radiation exposure; c) treatment with chemotherapy and/or radiotherapy; d) bone marrow failure; e) bone marrow transplantation; and f) cord blood transplantation. The method can include placental derived endothelial progenitor cells that are obtained from an allogeneic placenta from which; a) the fetal vascular lobules have been dissociated; b) the dissociated (homogenized) lobules of step a) are enzymatically digested with a preparation of approximately 2% collagenase, about 0.25% trypsin and about 0.1% DNAse in tissue culture medium; c) filtering the digestion product of step b) to remove particulates; d) obtaining a mononuclear cells from the filtered digestion product of step c) by density gradient centrifugation; e) plating the mononuclear cells on a collagen I-coated tissue culture plate; f) growing the mononuclear cells to confluency; g) detaching the confluent cells from the plate; and h) sorting the detached cells for expression of CD144 and substantially lack of expression of CD45. Said isolated placental vascular lobe endothelial progenitor cells can be expanded in vitro.

Further embodiments include a method of accelerating hematopoietic reconstitution by administration of placentally-derived endothelial progenitor cells. The method can be conducted wherein said placentally-derived endothelial progenitor cells are allogeneic to the recipient in need of hematopoietic reconstitution. Additionally, a method of accelerating hematopoietic reconstitution by administration of placentally-derived endothelial cells is provided herein. Said method of claim can be conducted wherein said placentally-derived endothelial cells are allogeneic to the recipient in need of hematopoietic reconstitution.

### DETAILED DESCRIPTION

This application provides compositions of isolated EPC and endothelial cell populations populations derived from fetal vascular lobules of a hemochorial placenta, particularly a hemochorial placenta from a human that are useful for the stimulation of hematopoietic reconstitution. In one embodiment of the invention the EPC express CD144, CD105, and/or CD31, either immediately upon isolation or after culturing. In certain aspects, EPC derived from placental tissue do not express CD45. In one embodiment of the invention, the stem and/or endothelial progenitor cells express CD144, CD105, and CD31 but do not express do not express CD45. Certain isolated EPC or endothelial cell populations of the invention can form capillary-like tubules when plated on a Matrigel substrate and can take up DiI-acetylated-low-density-lipoprotein.

In certain embodiments, the isolated stem and/or endothelial progenitor cell populations of the invention are prepared by homogenizing fetal vascular lobules from a full-term placenta; successively digesting the homogenized lobules with a preparation of about 2% collagenase, about 0.25% trypsin and about 0.1% DNAse, in tissue culture medium such as DMEM. The digestion product is then filtered to remove particulates, and mononuclear cells are obtained therefrom by density gradient centrifugation. The mononuclear cells can then be plated on collagen I-coated tissue culture plates and grown to confluency. Detached cells from the confluent plates are then sorted to obtain stem and/or progenitor cells that express of CD144 but lack of expression of CD45.

The present application also provides methods for treating patients in need of accelerated hematopoietic reconstitution, including patients exposed to radiation, chemotherapy, bone marrow transplant, cord blood transplant, or suffering from bone marrow failure.

### EXAMPLES

### Example 1

### Radioprotection by Placental Vascular Lobule EPC

Fetal vascular lobules are placed in a blender with HBSS and homogenized. The homogenate is centrifuged at 600.times.g for 6 minutes and washed three times with PBS. The pelleted cells are then digested with 2% collagenase in DMEM, 0.25% trypsin and 0.1% DNase in sequence. The resulting preparation is filtered and the mononuclear cell fraction (MNC) are isolated with Ficoll gradient centrifugation. Cells are washed 2 times in PBS.

Female BALB/c mice 6-8 weeks of age are irradiated twice with 575-600 cGy 3 hours apart using a J. L. Shepherd Co. Cesium irradiator. Placental vascular lobule EPC are diluted in 200 μl of modified HBSS at doses of 3×10(4), 1×10(4) and 1×10(3). Following the second dose of irradiation, donor cells are injected into the retroorbital plexus of recipients anesthetized with isoflurane. Irradiated control mice received 200 μl modified HBSS only. Recipient mice that had been maintained acidified water were switched to non-acidified water containing antibiotics (106 unit/liter Polymyxin B sulfate and 1.1 g/liter neomycin sulfate) and monitored daily over 60 days.

Peripheral blood is obtained from primary or secondary recipients by retroorbital puncture. Aliquots of 200 μl are analyzed for complete blood counts and platelet counts (Antech Diagnostics, Portland, Oreg.). For the determination of donor-derived hematopoiesis, peripheral blood is collected and nucleated cells were prepared by sedimenting erythrocytes in 2% Dextran (T-500) followed by hypotonic lysis. Cell pellets were washed and incubated with anti-CD45.1-FITC and anti-CD45.2-PE in combination with lineage specific markers for Tcells (CD3-APC), B-cells (B220-APC) or myelomonocytic cells (Mac-1-APC and Gr-1-APC) (BD Pharmingen). The co-expression of these cell surface antigens is determined by using a FACscan II and dead cells were excluded using scatter gates and propidium iodide. Up to 50 thousand events were analyzed to provide a sensitivity of 0.5%. Hemoglobin analysis was performed on peripheral blood isolated as described previously in the art. Approximately 70 μl of peripheral blood is collected from each recipient mouse, centrifuged and the pellets were lysed with 1× cystamine solution. Hemoglobin lysates are applied to a cellulose acetate plate (Helena Laboratories, Beaumont, Tex.) and electrophoresed at 300 volts for 30 minutes. Following electrophoresis, plates were stained with Ponceau S for 20 minutes, rinsed in deionized water, and destained in 2 changes of 7% glacial acetic acid prior to imaging.

A dose-dependent increase in survival is noted in animals receiving EPC as compared to controls. Acceleration of hematopoietic reconstitution is observed, as well as increased recovery of red blood cells and hemoglobin content.

### Example 2

### Augmentation of Cord-Blood Reconstitution after Nuclear Incident

A nuclear attack on a populated city occurs exposing 50 individuals to an estimated 10 Gy Eq of neutron and gamma irradiation. All 50 patients presented with symptoms of acute radiation syndrome including severe pancytopenia. Based on previous experiences (Nagayama, et al., 2002. Int J Hematol 76:157-164, which is incorporated by reference herein in its entirety), and the lack of sibling related donors or possibility of autotransplantation, the use of cord blood as a hematopoietic graft is performed after HLA-matching allowing for only one allele mismatch.

Pretransplantation conditioning consists of antithymocyte equine 3 globulin alone (2.5 mg/kg for 2 consecutive days), and GVHD prophylaxis consists of the combined use of cyclosporine A (CyA) and methylprednisolone (mPSL). Patients are administered 3.times.10.sup.7 nucleated cord blood cells per kilogram through intravenous infusion. All patients are administered filgrastim (neupogen) at a concentration of 10 .mu.g/kg/day for 14 days in order to accelerate leukocytic recovery. Of the 50 patients, 25 receive concurrently with filgrastim, a concentration of 1 million allogeneic placental vascular EPC/kg/day. The cells are prepared under GMP conditions based on the description of Example 1. At day 15 after cellular transplantation, 23% of patients treated with filgrastim alone have granulocytic counts of more than 500/mm.sup.3. In contrast, 100% of the patients receiving the combination of filgrastim and EPC have granulocytic counts of more than 500/mm.sup.3 by day 12 post transplantation. Chimeric hematopoiesis is observed at day 50 in 46% of patients treated with filgrastim alone, whereas 100% of patients receiving the combination had achieved this milestone. Additionally, opportunistic infections are predominantly associated with the patient group that received filgrastim alone. This example suggests the use of placental vascular EPC as an adjuvant agent to standard hematopoiesis stimulating regimens. Additionally, although GVHD is not observed in any of the patients in the prior example, most likely due to the low levels of cord blood cells administered, higher doses of cord blood cells can predispose to this. Accordingly, placental vascular lobule EPC can be used in combination with immune suppressive cytokines to preferential stimulate expansion of natural immune regulatory cell subsets.

One skilled in the art will appreciate that these methods and devices are and can be adapted to carry out the objects and obtain the ends and advantages mentioned, as well as those inherent therein. The methods, procedures, and devices described herein are presently representative of preferred embodiments and are exemplary and are not intended as limitations on the scope of the invention. Changes therein and other uses will occur to those skilled in the art which are encompassed within the spirit of the invention and are defined by the scope of the disclosure.

It is apparent to one skilled in the art that varying substitutions and modifications can be made to the invention disclosed herein without departing from the scope and spirit of the invention. Furthermore, those skilled in the art recognize that the aspects and embodiments of the invention set forth herein can be practiced separate from each other or in conjunction with each other. Therefore, combinations of separate embodiments are within the scope of the invention as disclosed herein.

1. A composition useful for accelerating reconstitution of the hematopoietic compartment after an insult to bone marrow function, said composition comprising of cells isolated from placental structure derived endothelial cells.

2. The composition of claim 1, wherein said cell isolated form placenta perivascular tissue expresses at a marker selected from a group of markers comprising: CD144, CD105, and CD31.

3. The composition of claim 1, wherein said placenta perivascular tissue is isolated from fetal vascular lobules of a hemochorial placenta.

4. The composition of claim 1, wherein said cells are prepared by: a) dissociating fetal vascular lobules from a full-term human placenta; b) successively digesting the homogenized lobules of step a) with a preparation of about 2% collagenase, about 0.25% trypsin and about 0.1% DNAse in tissue culture medium; c) filtering the digestion product of step b) to remove particulates; d) obtaining a mononuclear cells from the filtered digestion product of step c) by density gradient centrifugation; e) plating the mononuclear cells on a collagen I-coated tissue culture plate; f) growing the mononuclear cells to confluency; g) detaching the confluent cells from the plate; and h) sorting the detached cells for expression of CD144 and substantially lack of expression of CD45.

5. The composition of claim 4, wherein said cells isolated by enzymatic digestion of placenta are administered into a patient in need of treatment without an expansion step.

6. The composition of claim 5, wherein said cells take up DiI-acetylated-low [symbol] idensity-lipoprotein.

7. The composition of claim 1, wherein said cells constitute a population of cells containing endothelial progenitor cells.

8. A composition useful for acceleration of hematopoietic reconstitution, said composition comprising of endothelial cells and endothelial progenitor cells derived from fetal vascular lobules of a hemochorial placenta.

9. The composition of claim 8, wherein said endothelial progenitor cells substantially express at least one marker selected from: CD144, CD105, and CD31.

10. The composition of claim 9, wherein said endothelial progenitor cells lack substantial expression of a marker selected from a group of markers comprising of: a) CD14; and b) CD45.

11. The composition of claim 10, wherein said endothelial progenitor cells are capable of taking up DiI-acetylated-low-density-lipoprotein.

12. The composition of claim 10, wherein said endothelial precursor cells are manufactured by: a) dissociating fetal vascular lobules from a full-term human placenta; b) successively digesting the homogenized lobules of step a) with a preparation of about 2% collagenase, about 0.25% trypsin and about 0.1% DNAse in tissue culture medium; and c) filtering the digestion product of step b) to remove particulates; d) obtaining a cell preparation useful for administration.

13. The composition of claim 1, wherein said composition is administered intravenously prior to, at the moment of, or subsequent to exposure to an agent or plurality of agents causing destruction of hematopoietic tissue.

14. The composition of claim 1, wherein said composition is administered together with a growth factor capable of stimulating proliferation and/or differentiation of hematopoietic stem cells.

15. A method of augmenting hematopoiesis in a patient, said method consisting of: a) selecting a patient in need of therapy; b) obtaining a population of cells containing allogeneic placental derived endothelial progenitor cells; and c) infusing into said patient said population of allogeneic placental derived endothelial progenitor cells.

16. The method of claim 15, wherein said patient in need of therapy suffers from a disorder selected from a group of disorders comprising of: a) acute radiation syndrome; b) radiation exposure; c) treatment with chemotherapy and/or radiotherapy; d) bone marrow failure; e) bone marrow transplantation; and f) cord blood transplantation.

17. The method of claim 16, wherein said placental derived endothelial progenitor cells are obtained from an allogeneic placenta from which; a) the fetal vascular lobules have been dissociated; b) the dissociated (homogenized) lobules of step a) are enzymatically digested with a preparation of approximately 2% collagenase, about 0.25% trypsin and about 0.1% DNAse in tissue culture medium; c) filtering the digestion product of step b) to remove particulates; d) obtaining a mononuclear cells from the filtered digestion product of step c) by density gradient centrifugation;
e) plating the mononuclear cells on a collagen I-coated tissue culture plate; f) growing the mononuclear cells to confluency; g) detaching the confluent cells from the plate; and h) sorting the detached cells for expression of CD144 and substantially lack of expression of CD45.

18. The method of claim 17, wherein isolated placental vascular lobe endothelial progenitor cells are expanded in vitro.
